(19) United States
(12) Patent Application Publication
(10) Pub. No.: US 2020/0372818 A1
(43) Pub. Date: Nov. 26, 2020

(54) STATE DETERMINATION APPARATUS, STATE DETERMINATION METHOD, AND COMPUTER PROGRAM

(71) Applicants: HONDA MOTOR CO., LTD., Tokyo (JP); KYUSHU UNIVERSITY, NATIONAL UNIVERSITY CORPORATION, Fukuoka-shi (JP)

(72) Inventors: Tadahiro Kubota, Wako-shi (JP); Tomohiro Imai, Wako-shi (JP); Shigekazu Higuchi, Fukuoka-shi (JP); Yuki Motomura, Fukuoka-shi (JP); Yeon-Kyu Kim, Fukuoka-shi (JP); Kosuke Okusa, Fukuoka-shi (JP); Midori Motoi, Fukuoka-shi (JP); Yuki Ikeda, Fukuoka-shi (JP); Sayuri Hayashi, Fukuoka-shi (JP)

(21) Appl. No.: 15/930,479

(22) Filed: May 13, 2020

(30) Foreign Application Priority Data
May 22, 2019 (JP) 2019-095947

(51) Int. Cl. G09B 7/00; A61B 5/02; A61B 5/00; G06F 16/906

(52) U.S. Cl. CPC G09B 7/00; G06F 16/906; A61B 5/0004; A61B 5/02

(57) ABSTRACT

A state determination apparatus includes: an acquisition unit acquiring first questionnaire information indicating an answer to a first questionnaire in which the answer does not indicate an individuality of an answerer, first physiological information indicating a physiological state of the answerer, second questionnaire information indicating an answer to a second questionnaire in which the answer indicates an individuality of the answerer, and second physiological information indicating a physiological state of the answerer; a membership cluster estimation unit estimating to which cluster a determination target person belongs on the basis of the second physiological information or the second questionnaire information provided by the target person, and a membership condition; and a state determination unit determining a state of the target person on the basis of state relationship information indicating a relationship between the first questionnaire information and the first physiological information in a cluster as an estimation result, and the first physiological information of the target person, in which a combination of the first physiological information and the first questionnaire information indicates individuality.

### CROSS-REFERENCE TO RELATED APPLICATION

Priority is claimed on Japanese Patent Application No. 2019-095947, filed on May 22, 2019, the contents of which are incorporated herein by reference.

### BACKGROUND

### Field of the Invention

The present invention relates to a state determination apparatus, a state determination method, and a computer program.

### Background

There are techniques (refer to Japanese Unexamined Patent Application, First Publication No. H10-272959 and Japanese Unexamined Patent Application, First Publication No. 2013-13542) of determining a state of a person (hereinafter, referred to as a “person's state”) such as drowsiness or an emotional state on the basis of physiological information of the person.

A person's state is generally changed by two factors. First, a person's state is changed by a neural response that occurs inside the person due to an external stimulus. Second, a person's state is changed by an experience acquired through acquired learning.

### SUMMARY

Regardless of which factor changes a person's state, there are individual differences in a person's state or a change of a person's state for the same change, and variations among individuals are great. Thus, it is difficult to determine the state of a specific person with high accuracy according to a unitary method. A unitary method is a method of determining person's states on the basis of the same formula with respect to all individuals.

An aspect of the present invention is a technique relating to determination accuracy in a technique of determining a person's state, and an object thereof is to provide a technique of suppressing deterioration in determination accuracy caused by individual differences in a change of a person's state.

According to a first aspect of the present invention, there is provided a state determination apparatus including an acquisition unit acquiring first physiological information, and second physiological information or second questionnaire information with respect to a determination target person who is a determination target among first questionnaire information that is information indicating an answer of an answerer to a first questionnaire in which the answer does not indicate an individuality of the answerer, the first physiological information that is information indicating a physiological state of the answerer to the first questionnaire and not indicating the individuality of the answerer to the first questionnaire, the second questionnaire information that is information indicating an answer of an answerer to a second questionnaire in which the answer indicates an individuality of the answerer, and the second physiological information that is information indicating a physiological state of the answerer to the second questionnaire and indicating an individuality of the answerer to the second questionnaire; a membership cluster estimation unit estimating to which cluster the determination target person belongs among a plurality of clusters forming a population of answerers based on the second physiological information or the second questionnaire information, and a membership condition according to the second physiological information or the second questionnaire information; an answer estimation unit estimating an answer of the determination target person to the first questionnaire based on state relationship information indicating a relationship between the first questionnaire information and the first physiological information, predefined for each of the plurality of clusters, the state relationship information corresponding to an estimation result of the membership cluster estimation unit, and the first physiological information provided by the determination target person; and a state determination unit determining a state of the determination target person based on the answer estimated by the answer estimation unit, in which the first physiological information and the first questionnaire information are information indicating an individuality through a combination of the first physiological information and the first questionnaire information.

According to a second aspect of the present invention, the state determination apparatus of the first aspect may further include a state relationship information acquisition unit acquiring state relationship information for each cluster based on the first physiological information and the first questionnaire information provided by a plurality of population constituents each forming the population; and a first update unit updating the state relationship information based on the answer and the first physiological information provided by the determination target person in a case where an update condition that is a predetermined condition related to the answer estimated by the answer estimation unit is satisfied.

According to a third aspect of the present invention, the state determination apparatus of the second aspect may further include a clustering unit classifying the population constituents into a plurality of clusters based on the first physiological information and the first questionnaire information of the plurality of population constituents; and a second update unit causing the clustering unit to classify the population constituents and the determination target person into a plurality of clusters based on the first physiological information and the first questionnaire information of the plurality of population constituents, the first physiological information of the determination target person, and the state relationship information corresponding to an estimation result of the membership cluster estimation unit, in a case where the update condition is satisfied.

According to a fourth aspect of the present invention, there is provided a state determination method including acquiring first physiological information, and second physiological information or second questionnaire information with respect to a determination target person who is a determination target among first questionnaire information that is information indicating an answer of an answerer to a first questionnaire in which the answer does not indicate an individuality of the answerer, the first physiological information that is information indicating a physiological state of the answerer to the first questionnaire and not indicating the individuality of the answerer to the first questionnaire, the second questionnaire information that is information indicating an answer of an answerer to a second questionnaire in which the answer indicates an individuality of the answerer, and the second physiological information that is information indicating a physiological state of the answerer to the second questionnaire and indicating an individuality of the answerer to the second questionnaire; estimating to which cluster the determination target person belongs among a plurality of clusters forming a population of answerers based on the second physiological information or the second questionnaire information, and a membership condition according to the second physiological information or the second questionnaire information; estimating an answer of the determination target person to the first questionnaire based on state relationship information indicating a relationship between the first questionnaire information and the first physiological information, predefined for each of the plurality of clusters, the state relationship information corresponding to a result of the estimation regarding a membership cluster, and the first physiological information provided by the determination target person; and determining a state of the determination target person based on the estimated answer, in which the first physiological information and the first questionnaire information are information indicating an individuality through a combination of the first physiological information and the first questionnaire information.

According to a fifth aspect of the present invention, there is provided a non-transitory computer readable recording medium including a computer program for causing a computer to function as the state determination apparatus according to the first to third aspects.

According to the first, second, third, fourth, and fifth aspects, the state determination apparatus determines to which cluster a determination target person belongs among a plurality of clusters that are results clustered in advance on the basis of the first physiological information and the first questionnaire information provided by a plurality of providers. The state determination apparatus determines a state of a determination target person on the basis of the state relationship information for each cluster indicated by a determination result and the second physiological information or the second questionnaire information provided by the determination target person. As mentioned above, the state determination apparatus does not determine a state of a person according to a one-dimensional method, but determines a state of a determination target person according to state relationship information corresponding to a cluster to which the determination target person belongs. Thus, according to the first, second, third, fourth, and fifth aspects, the state determination apparatus can suppress deterioration in determination accuracy due to individual differences in a change of a state of a person.

According to the second aspect, the state determination apparatus updates the state relationship information on the basis of the answer and the first physiological information provided by the determination target person in a case where an update condition that is a predetermined condition related to the answer estimated by the answer estimation unit is satisfied. Thus, it is possible to improve the accuracy of determination of determining a state of a determination target person.

According to the third aspect, the state determination apparatus executes clustering again on the basis of the first physiological information provided by the determination target person and the state relationship information corresponding to an estimation result from the membership cluster estimation unit. Thus, it is possible to improve the accuracy of determination of determining a state of a determination target person.

### BRIEF DESCRIPTION OF THE DRAWINGS

FIG. 1 is a diagram showing an example of a functional configuration of a state determination apparatus of an embodiment.

FIG. 2 is a diagram showing a membership condition in the embodiment.

FIG. 3 is a flowchart showing an example of a flow of a process executed by the state determination apparatus in the embodiment.

FIG. 4 is a diagram showing an example of a test result showing the accuracy of a membership condition acquired by the state determination apparatus of the embodiment.

FIGS. 5A to 5E are first diagrams of test results showing an example of first questionnaire information in the embodiment.

FIGS. 6A to 6E are second diagrams of test results showing an example of the first questionnaire information in the embodiment.

FIGS. 7A to 7D are third diagrams of test results showing an example of the first questionnaire information in the embodiment.

FIG. 8 is a diagram of a test result showing an example of an execution result of a clustering process in the embodiment.

FIG. 9 is a first diagram of a test result showing an example of an execution result of a state information acquisition process in the embodiment.

FIG. 10 is a second diagram of a test result showing an example of an execution result of the state information acquisition process in the embodiment.

FIG. 11 is a diagram showing examples of test results showing the accuracy with which the state determination apparatus of the embodiment determines a state of a determination target person and the accuracy with which an apparatus of the related art determines a state of a determination target person.

### DESCRIPTION OF EMBODIMENTS

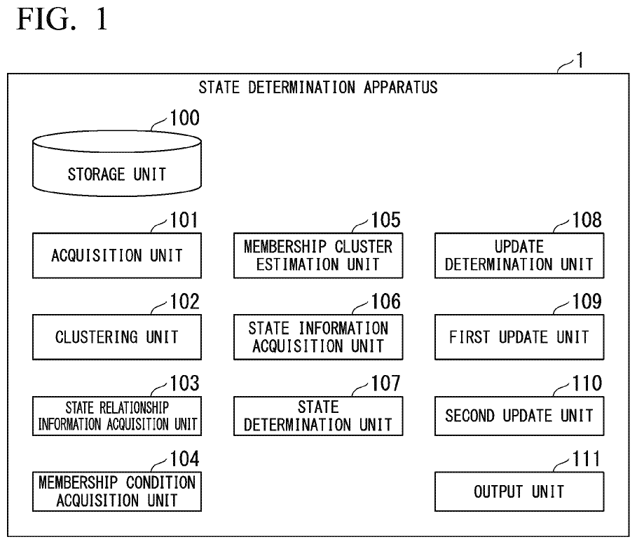

FIG. 1 is a diagram showing an example of a functional configuration of a state determination apparatus 1 of an embodiment. The state determination apparatus 1 determines a state of a determination target person. The state of the determination target person is a state such as drowsiness or an emotional state.

The state determination apparatus 1 includes a processor such as a central processing unit (CPU), a memory, a storage unit 100, and the like connected to each other via a bus, and executes a program. The state determination apparatus 1 functions as an apparatus including an acquisition unit 101, a clustering unit 102, a state relationship information acquisition unit 103, a membership condition acquisition unit 104, a membership cluster estimation unit 105, a state information acquisition unit 106, a state determination unit 107, an update determination unit 108, a first update unit 109, a second update unit 110, and an output unit 111 by executing the program.

The acquisition unit 101 is configured to include an input device such as a mouse, a keyboard, or a touch panel. The acquisition unit 101 may be configured as an interface connecting the input device to the state determination apparatus. The acquisition unit 101 receives input of various pieces of information on the state determination apparatus. The various pieces of information input to the acquisition unit 101 include first physiological information provided from a determination target person, and first questionnaire information and first physiological information provided from a plurality of persons other than the determination target person. Hereinafter, a person other than a determination target person will be referred to as a population constituent.

The first questionnaire information is information indicating an answer of an answerer to a first questionnaire.

Questions in the first questionnaire include questions regarding a state of an answerer. The first questionnaire information is information indicating answers of an answerer to the questions in the first questionnaire. A question in the first questionnaire may be, for example, a question for personality test. In this case, the first questionnaire information is information indicating an answer of an answerer to the question for personality test. The first questionnaire information may indicate an answer of an answerer to the first questionnaire in any form as long as the answerer of the answerer to the first questionnaire is indicated in a computer readable form. The first questionnaire information may be, for example, a value indicating an answer of an answerer to the first questionnaire. The value indicating an answer of an answerer to the first questionnaire may be 1, for example, in a case where the answer of the answerer to the first questionnaire is yes, and may be 0 in a case where the answer of the answerer to the first questionnaire is no. The first questionnaire information may indicate an answer of an answerer to the first questionnaire in the form of visual analogue scale (VAS).

The first physiological information is information indicating a first physiological state of an answerer to a question in the first questionnaire. The first physiological state is a physiological state in which a changes has a correlation with a change of an answer of an answerer to the first questionnaire. The first physiological information is acquired from an answerer. The first physiological information may be any information as long as the information indicates the first physiological state. The first physiological information may be, for example, a value (hereinafter, referred to as a “first physiological value”) indicating the first physiological state. The first physiological value may be, for example, a physical quantity representing an electrocardiogram waveform, a pulse, a fingertip pulse, an earlobe pulse, a skin temperature, a skin resistance, a respiration, a pupil diameter, a visual line, a blink, or a facial electromyograph. The physical quantity representing an electrocardiogram waveform may be, for example, the maximum value of amplitude of an electrocardiogram waveform, and may be a frequency thereof. A plurality of physical quantities indicated by values of the physiological information may be two or more physical quantities among a physical quantity representing an electrocardiogram waveform, a pulse, a fingertip pulse, an earlobe pulse, a skin temperature, a skin resistance, a respiration, a pupil diameter, a visual line, a blink, or a facial electromyograph.

The first physiological information may be information regarding a physiological state acquired from an answerer to the first questionnaire when the answerer is answering the first questionnaire.

Each of the first physiological information and the first questionnaire information is information not indicating an individuality, and the first physiological information and the first questionnaire information are information indicating an individuality of a provider through a combination thereof.

Not indicating an individuality involves an individual difference not being represented in a relationship between a change of information and a change of a state of a person such as drowsiness or an emotional state. Indicating an individuality involves an individual difference being represented in a relationship between a change of information and a change of a state of a person such as drowsiness or an emotional state. Since the first questionnaire information is information not indicating an individuality, the first questionnaire is a questionnaire to which an answer does not indicate the individuality of an answerer.

The various pieces of information input to the acquisition unit 101 include second questionnaire information and second physiological information provided from a determination target person, and second questionnaire information and second physiological information provided from a population constituent. The second questionnaire information is information indicating an answer of an answerer to a second questionnaire. The second questionnaire is different from the first questionnaire in terms of some or all questions.

A question in the second questionnaire may be, for example, a question for personality test. In this case, the second questionnaire information is information indicating an answer of an answerer to the question for personality test. The second questionnaire information may indicate an answer of an answerer to the second questionnaire in any form as long as the answer of the answerer to the second questionnaire is indicated in a computer readable form. The second questionnaire information may be, for example, a value indicating an answer of an answerer to the second questionnaire. The value indicating an answer of an answerer to the second questionnaire may be 1, for example, in a case where the answer of the answerer to the second questionnaire is yes, and may be 0 in a case where the answer of the answerer to the second questionnaire is no. The second questionnaire information may indicate an answer of an answerer to the second questionnaire in the form of visual analogue scale (VAS).

The second physiological information is information indicating a second physiological state of an answerer. The second physiological state is a physiological state during normal times or at rest. The second physiological information is acquired from an answerer. The second physiological information may be any information as long as the information indicates the second physiological state. The second physiological information may be, for example, a value (hereinafter, referred to as a “second physiological value”) indicating the second physiological state. The second physiological value may be, for example, a physical quantity representing an electrocardiogram waveform, a pulse, a fingertip pulse, an earlobe pulse, a skin temperature, a skin resistance, a respiration, a pupil diameter, a visual line, a blink, or a facial electromyograph. The physical quantity representing an electrocardiogram waveform may be, for example, the maximum value of amplitude of an electrocardiogram waveform, and may be a frequency thereof. A plurality of physical quantities indicated by values of the physiological information may be two or more physical quantities among a physical quantity representing an electrocardiogram waveform, a pulse, a fingertip pulse, an earlobe pulse, a skin temperature, a skin resistance, a respiration, a pupil diameter, a visual line, a blink, or a facial electromyograph.

The second physiological information may be information regarding a physiological state acquired from an answerer to the second questionnaire when the answerer is answering the second questionnaire.

Each of the second physiological information and the second questionnaire information is information indicating an individuality. The information indicating an individuality is information in which there is an individual difference in a relationship between a change of information and a change of a state of a person such as drowsiness or an emotional state. Specifically, the second physiological information is information in which there is an individual difference in a relationship between a change of a state of a person such as drowsiness or an emotional state of a provider of the second physiological information and a change of a physiological state. Specifically, the second questionnaire information is information in which there is an individual difference in a relationship between a change of a state of a person such as drowsiness or an emotional state of an answerer of the second questionnaire information and a change of an answer to the second questionnaire. Since the second questionnaire information is information indicating an individuality, the second questionnaire is a questionnaire to which an answer indicates the individuality of an answerer.

The second physiological state and a question in the second questionnaire are information that enable providers of the second physiological information and the second questionnaire information to be classified into clusters corresponding to clustering results from the clustering unit 102 which will be described later.

### (Detailed Description of Information not Indicating Individuality and Information Indicating Individuality)

Here, information not indicating an individuality and information indicating an individuality will be described in detail. As described above, the first physiological information and the first questionnaire information are information not indicating an individuality alone but indicating an individuality through a combination thereof. Hereinafter, a description will be made of information indicating an individuality by exemplifying a case where the first physiological information is a heartbeat, and the first questionnaire information is a value indicating the degree of comfort or discomfort. Heartbeats generally tend to decrease when an uncomfortable stimulus is given to a provider of the first physiological information. For example, the heartbeats decrease to about ten beats/minute for three seconds. Thus, the event that “the heartbeats decrease to about ten beats/minute for three seconds” occurs for anyone. The event that “the degree of discomfort of about 80 was felt” or the event that “there is the degree of drowsiness of about 70” is also an event occurring for anyone. As mentioned above, each of the first physiological information and the first questionnaire information is information not indicating an individuality. However, the event that “heartbeats decrease to ten beats/minute for three seconds at the degree of 80” may or may not occur depending on persons. Thus, the event has an individual difference.

The clustering unit 102 which will be described later clusters providers of the first physiological information and the first questionnaire information according to an event having an individual difference.

Next, the second physiological information and the second questionnaire information will be described. Each of the second physiological information and the second questionnaire information is information indicating an individuality. The second physiological information is, for example, an average heart rate in a period of about two minutes to ten minutes or blood pressure in a case where a state of a person is not changed as much as possible at rest or the like. The average heart rate or the blood pressure has a great individual difference, and, for example, a heart rate of 70 differs for each person. As mentioned above, the second physiological information is information indicating an individuality.

The second questionnaire information is information that cannot be acquired as the second physiological information, and is information in which an individual difference in, for example, a person's cognitive ability, sensitivity to emotions, mood characteristics, behavioral characteristics, empathy for others, or logical cognitive ability to explain a phenomenon is represented. As mentioned above, the second questionnaire information is information indicating an individuality other than information indicating a physiological state. Hitherto, information not indicating an individuality and information indicating an individuality have been described above in detail.

The acquisition unit 101 outputs the input various pieces of information to the clustering unit 102, the state relationship information acquisition unit 103, the membership condition acquisition unit 104, the membership cluster estimation unit 105, the state information acquisition unit 106, the state determination unit 107, the first update unit 109, the second update unit 110, and the output unit 111.

The clustering unit 102 clusters (classifies) providers providing the first questionnaire information and the first physiological information to form a plurality of clusters on the basis of the first questionnaire information and the first physiological information. The providers of the first questionnaire information and the first physiological information are answerers to questions in the first questionnaire, and are answerers providing the first physiological information. The clustering unit 102 may cluster providers according to any clustering method as long as the providers of the first questionnaire information and the first physiological information can be clustered to form a plurality of clusters on the basis of the first questionnaire information and the first physiological information. The clustering unit 102 may perform clustering according to, for example, a k-means method. The clustering unit 102 may perform clustering according to, for example, a model-based clustering method in which data is assumed to conform to a statistical distribution. The clustering unit 102 may perform clustering according to, for example, a linear regression method. The clustering unit 102 may perform clustering according to, for example, a time-series analysis method.

The clustering unit 102 may normalize the first questionnaire information and the first physiological information in clustering. The first questionnaire information and the first physiological information are normalized, and thus variations caused by individual differences therein can be reduced. A result of clustering performed by the clustering unit 102 is recorded in the storage unit 100.

Hereinafter, the first physiological information input to the clustering unit 102 will be referred to as input physiological information. Hereinafter, the first questionnaire information input to the clustering unit 102 will be referred to as input questionnaire information.

The state relationship information acquisition unit 103 acquires a state equation relationship information for each cluster formed by the clustering unit 102. The state relationship information is information representing a relationship between the first questionnaire information and the first physiological information in corresponding clusters. The state relationship information is an equation (hereinafter, referred to as a “state equation”) representing a relationship between the first questionnaire information and the first physiological information in corresponding clusters. The state equation may the same even in a case where clusters are different from each other, and may differ in a case where clusters are different from each other. The state equation is, for example, a polynomial (hereinafter, referred to as a “state polynomial”) represented by the following Equation (1).

Y=a×X1+b×X2+c×X3 (1)

In Equation (1), Y is a value of the state equation, and represents the first questionnaire information (that is, an answer of an answerer to the first questionnaire). The first questionnaire information includes a question about a state of an answerer, and thus the value Y of the state equation represents a subjective evaluation of a state of the answerer to the first questionnaire. Hereinafter, the value Y of the state equation will be referred to as a state value. In Equation (1), a, b, and c represent coefficients in the state polynomial. Values of the coefficients a, b, and c of Equation (1) in the state equation may or may not be the same for each cluster. In Equation (1), X1, X2, and X3 are variables representing the first physiological information. In other words, the first physiological values are assigned to X1, X2, and X3. X1, X2, and X3 may be any variables as long as the variables represent the first physiological state. X1, X2, and X3 may be variables representing a value of, for example, a physical quantity representing an electrocardiogram waveform, a pulse, a fingertip pulse, an earlobe pulse, a skin temperature, a skin resistance, a respiration, a pupil diameter, a visual line, a blink, or a facial electromyograph.

The state polynomial may not necessarily be a polynomial having three terms, and may be an equation having any number of terms in the form of a polynomial. The state polynomial may be, for example, a polynomial having four or more terms, and may be a polynomial having two terms. The state polynomial may be any polynomial in the form of a polynomial, and may not necessarily be a linear polynomial. The state polynomial may be a nonlinear polynomial.

The predetermined method may be any method as long as a state of a target person can be estimated according to the method, and may be, for example, regression or classification such as a decision tree, a random forest, a support vector, and deep learning that are machine learning.

In the state relationship information acquisition unit 103, in a case where the predetermined method is a machine learning method, for example, a criterion variable is the first questionnaire information, and an explanatory variable is the second physiological information or the second questionnaire information.

The state relationship information acquisition unit 103 acquiring the state equation involves, for example, determining values of coefficients of a state polynomial. The state relationship information acquired by the state relationship information acquisition unit 103 is recorded in the storage unit 100.

Hereinafter, for simplification of description, state relationship information is assumed to be a state equation. Hereinafter, for simplification of description, the state equation is assumed to be the state polynomial represented by Equation (1).

The membership condition acquisition unit 104 acquires a membership condition according to a predetermined method on the basis of the second questionnaire information, the second physiological information, and the clustering result from the clustering unit 102. The membership condition is a condition in which a result of clustering a population constituent on the basis of the first questionnaire information and the first physiological information is the same as a result of clustering a population constituent on the basis of the second questionnaire information and the second physiological information.

The predetermined method may be any method as long as a state of a target person can be estimated according to the method, and may be regression or classification such as a decision tree, a random forest, a support vector, and deep learning that are machine learning.

The membership condition is, for example, a decision tree for deciding to which cluster a provider of the second questionnaire information and the second physiological information belongs among respective clusters determined by the clustering unit 102 on the basis of the second questionnaire information and the second physiological information.

In the membership condition acquisition unit 104, in a case where the predetermined method is a machine learning method, for example, a criterion variable is a cluster determined by the clustering unit 102, and an explanatory variable is the second physiological information or the second questionnaire information.

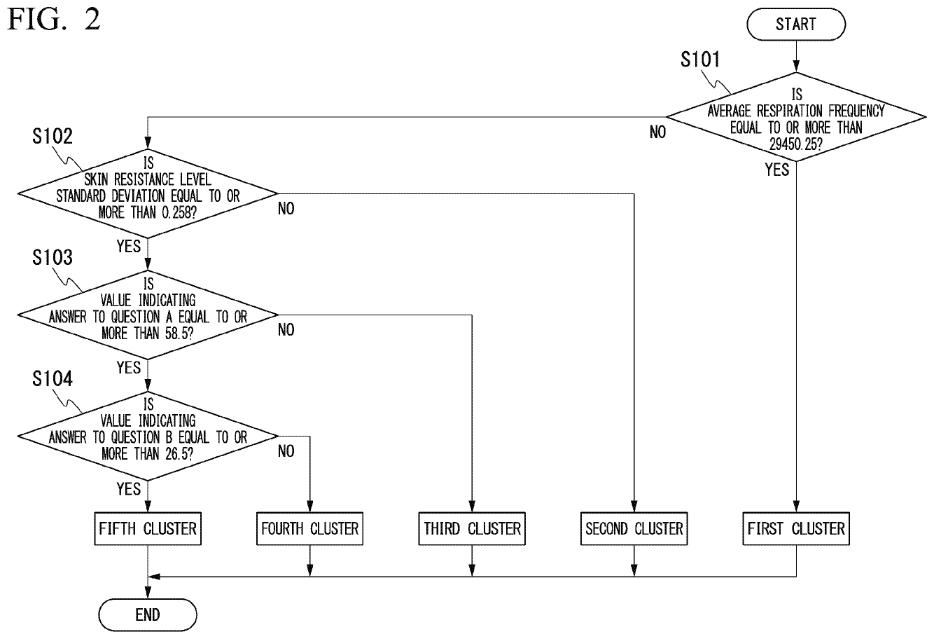

FIG. 2 is a diagram showing a membership condition in the embodiment. FIG. 2 shows a decision tree.

FIG. 2 shows an example of a decision tree obtained through tests. FIG. 2 shows an example of a decision tree in a case where providers of the first questionnaire information and the first physiological information are classified into five clusters such as a first cluster to a fifth cluster by the clustering unit 102.

A cluster to which a provider of the second questionnaire information and the second physiological information belongs is determined according to the decision tree shown in FIG. 2. Specifically, first, it is determined whether or not an average respiration frequency is equal to or more than 29450.25 (step S101). The average respiration frequency is an example of the second physiological information. In step S101, in a case where the average respiration frequency is equal to or more than 29450.25 (step S101: YES), a cluster to which the provider of the second questionnaire information and the second physiological information belongs is determined as being the first cluster.

On the other hand, in step S101, in a case where the average respiration frequency is less than 29450.25 (step S101: NO), it is determined whether or not a skin resistance level standard deviation is equal to or more than 0.258 (step S102). The skin resistance level standard deviation is an example of the second physiological information. In step S102, in a case where the skin resistance level standard deviation is less than 0.258 (step S102: NO), a cluster to which the provider of the second questionnaire information and the second physiological information belongs is determined as being the second cluster.

On the other hand, in step S102, in a case where the skin resistance level standard deviation is equal to or more than 0.258 (step S102: YES), it is determined whether or not a value indicating an answer to a question A that is one of questions in the second questionnaire is equal to or more than 58.5 (step S103). In FIG. 2, the value indicating an answer to the question A is a value represented according to the Toronto Alexithymia Scale. The value indicating an answer to the question A is an example of the second questionnaire information. In step S103, in a case where the value indicating the answer to the question A is less than 58.5 (step S103: NO), a cluster to which the provider of the second questionnaire information and the second physiological information belongs is determined as being the third cluster.

On the other hand, in step S103, in a case where the value indicating the answer to the question A is equal to or more than 58.5 (step S103: YES), it is determined whether or not a value indicating an answer to a question B that is one of questions in the second questionnaire is equal to or more than 26.5 (step S104). In FIG. 2, the question B is a question about physical factor cognitive tendencies. In FIG. 2, the value indicating an answer to the question B is a value represented according to the systemizing quotient. The value indicating an answer to the question B is an example of the second questionnaire information. In step S104, in a case where the value indicating the answer to the question B is less than 26.5 (step S104: NO), a cluster to which the provider of the second questionnaire information and the second physiological information belongs is determined as being the fourth cluster.

On the other hand, in step S104, in a case where the value indicating the answer to the question B is equal to or more than 26.5 (step S104: YES), a cluster to which the provider of the second questionnaire information and the second physiological information belongs is determined as being the fifth cluster.

The membership condition shown in FIG. 2 is a condition based on the second questionnaire information and the second physiological information, but the membership condition is not necessarily required to be based on the second questionnaire information and the second physiological information. The membership condition may be based on only the second questionnaire information, and may be based on only the second physiological information.

FIG. 1 is referred to again. The membership cluster estimation unit 105 estimates to which cluster a determination target person belongs among a plurality of predetermined clusters on the basis of the second physiological information and the second questionnaire information provided by the determination target person, and the membership condition. The membership cluster estimation unit 105 estimates to which cluster the determination target person belongs among a plurality of predetermined clusters forming a population of answerers on the basis of the second physiological information and the second questionnaire information provided by the determination target person, and the membership condition.

The state information acquisition unit 106 acquires state information on the basis of the first physiological information of the determination target person and state relationship information corresponding to the cluster estimated by the membership cluster estimation unit 105. The state information is information indicating a state of the determination target person. The state information is, for example, a state value. In other words, the state information is, for example, a value indicating a state of the determination target person. The state of the determination target person indicated by the state information is, for example, an emotion of the determination target person.

Hereinafter, the state relationship information corresponding to the cluster estimated by the membership cluster estimation unit 105 will be referred to as corresponding cluster state relationship information. The state relationship information corresponding to the cluster estimated by the membership cluster estimation unit 105 is state relationship information acquired with respect to the cluster estimated by the membership cluster estimation unit 105 among pieces of state relationship information acquired by the state relationship information acquisition unit 103.

The state information acquisition unit 106 acquiring state information involves the state information acquisition unit 106 calculating a state value on the basis of, for example, the first physiological information of a determination target person and a corresponding cluster state equation. The corresponding cluster state equation is a state equation corresponding to the cluster estimated by the membership cluster estimation unit 105. The state equation corresponding to the cluster estimated by the membership cluster estimation unit 105 is a state equation acquired with respect to the cluster estimated by the membership cluster estimation unit 105 among state equations acquired by the state relationship information acquisition unit 103.

The state information acquisition unit 106 assigns, for example, the first physiological value of the determination target person to the corresponding cluster state equation, and acquires an assigned result as a state value.

Since the state information is information indicating a state of the determination target person, the state information acquired by the state information acquisition unit 106 is an estimation result of an answer to the first questionnaire, given by the determination target person. Therefore, the state information acquisition unit 106 acquiring the state information involves the state information acquisition unit 106 estimating an answer of the determination target person to the first questionnaire.

The state determination unit 107 determines a state of the determination target person on the basis of the state information acquired by the state information acquisition unit 106. For example, in a case where the state information is a state value, and the state value is equal to or greater than a predetermined value, the state determination unit 107 determines that the determination target person is in a drowsy state.

The update determination unit 108 determines whether or not an update condition is satisfied. The update condition may be any condition as long as the condition is a condition related to the state information acquired by the state information acquisition unit 106.

The update condition may be, for example, a condition that a state value calculated by the state information acquisition unit 106 changes the average value of state values in a corresponding cluster to a predetermined value or greater.

The update condition may be, for example, a condition that a state value calculated by the state information acquisition unit 106 is smaller than a predetermined value.

In a case where the update condition is satisfied, the first update unit 109 updates the corresponding cluster state relationship information on the basis of the state value and the first physiological information of the determination target person according to a predetermined algorithm related to update of the state relationship information. Hereinafter, the predetermined algorithm related to update of the state relationship information will be referred to as a state relationship information update algorithm. The state relationship information update algorithm may be any method as long as a state of a target person can be estimated according to the method, and may be regression or classification such as a decision tree, a random forest, a support vector, and deep learning that are machine learning.

The first update unit 109 updates a value of a coefficient of the corresponding cluster state equation when the update is performed, for example, in a case where the state information is a state equation.

In a case where the update determination unit 108 determines that the update condition is satisfied, the second update unit 110 causes the clustering unit 102 to execute clustering with updated physiological information as input physiological information and with updated questionnaire information as input questionnaire information. The updated physiological information is information obtained by adding the first physiological information provided by a determination target person to the first physiological information provided by a population constituent. The updated questionnaire information is information obtained by adding determination target person questionnaire information to the first questionnaire information provided by a population constituent. The determination target person questionnaire information is the first questionnaire information acquired on the basis of the first physiological information provided by a determination target person and the corresponding cluster state relationship information. For example, the determination target person questionnaire information is a value obtained by assigning the first physiological information provided by the determination target person to the corresponding cluster state equation.

The output unit 111 is configured to include a display device such as a cathode ray tube (CRT) display, a liquid crystal display, or an organic electroluminescence (EL) display. The output unit 111 may be configured as an interface connecting the display device to the state determination apparatus.

The output unit 111 outputs an acquisition result in the state information acquisition unit 106 in a case where the update condition is not satisfied.

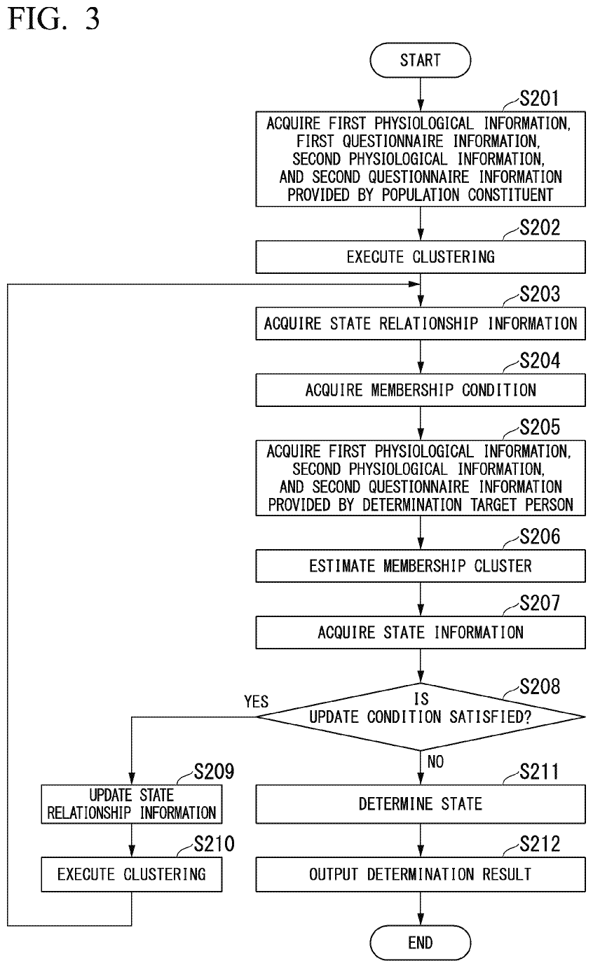

FIG. 3 is a flowchart showing an example of a flow of a process executed by the state determination apparatus 1 in the embodiment.

The acquisition unit 101 acquires the first physiological information, the first questionnaire information, the second physiological information, and the second questionnaire information provided by a plurality of population constituents (step S201). The acquisition unit 101 acquires the first physiological information, the first questionnaire information, the second physiological information, and the second questionnaire information when the first physiological information, the first questionnaire information, the second physiological information, and the second questionnaire information provided by the plurality of population constituents are input to the acquisition unit 101.

The clustering unit 102 clusters the population constituents to form a plurality of clusters on the basis of the first physiological information and the first questionnaire information acquired in step S201 (step S202). The state relationship information acquisition unit 103 acquires state relationship information in each cluster formed in step S202 (step S203).

The membership condition acquisition unit 104 acquires a membership condition on the basis of the second questionnaire information and the second physiological information acquired in step S201 and the clustering result in step S202 (step S204).

The acquisition unit 101 acquires the first physiological information, the second physiological information, and the second questionnaire information provided by a determination target person (step S205). For example, the acquisition unit 101 acquires the first physiological information, the second physiological information, and the second questionnaire information when the first physiological information, the second physiological information, and the second questionnaire information provided by the determination target person are input to the acquisition unit 101.

The membership cluster estimation unit 105 estimates to which cluster the determination target person belongs among the plurality of clusters formed in step S102 on the basis of the second physiological information and the second questionnaire information acquired in step S205 and the membership condition (step S206).

The state information acquisition unit 106 acquires state information on the basis of the first physiological information of the determination target person and an estimation result in step S206 (step S207).

The update determination unit 108 determines whether or not an update condition is satisfied (step S208).

In a case where the update condition is satisfied (step S208: YES), the first update unit 109 updates state relationship information of a cluster to which the determination target person is determined as belonging in step S206 on the basis of the state information and the first physiological information of the determination target person (step S209).

After step S209, the second update unit 110 causes the clustering unit 102 to execute clustering on the basis of updated physiological information and updated questionnaire information (step S210).

The process in step S210 is executed, and then the flow returns to process in step S203.

On the other hand, in step S208, in a case where the update condition is not satisfied (step S208: NO), the state determination unit 107 determines a state of the determination target person on the basis of the state information acquired in step S207 (step S211). The output unit 111 outputs a determination result in step S211 (step S212). After step S212, the process is finished.

Hereinafter, a process in which the state determination apparatus 1 clusters a provider of the first physiological information and the first questionnaire information on the basis of a plurality of pieces of first physiological information and the first questionnaire information will be referred to as a clustering process. The clustering process is the process in step S202.

Hereinafter, a process in which the state determination apparatus 1 acquires state relationship information will be referred to as a state relationship information acquisition process. The state relationship information acquisition process is a process in step S203. Hereinafter, a process in which the state determination apparatus 1 acquires a membership condition will be referred to as a membership condition acquisition process. The membership condition acquisition process is the process in step S204.

Hereinafter, a process in which the state determination apparatus 1 determines to which cluster a determination target person belongs among a plurality of clusters that are results of the clustering process on the basis of the second physiological information and the second questionnaire information will be referred to as a membership cluster determination process. The membership cluster determination process is the process in step S206.

Hereinafter, a process in which the state determination apparatus 1 acquires state information on the basis of state relationship information corresponding to a cluster indicated by a determination result of the membership cluster determination process will be referred to as a state information acquisition process. The state information acquisition process is the process in step S207.

### (Test Result)

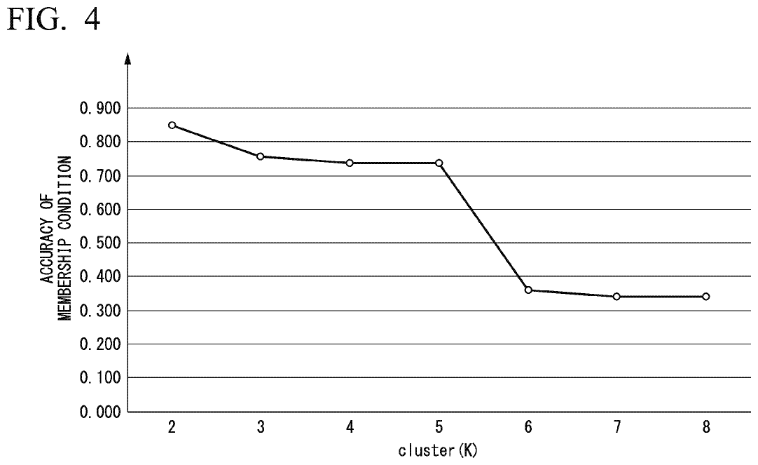

FIG. 4 is a diagram showing an example of a test result showing the accuracy of a membership condition acquired by the state determination apparatus 1 of the embodiment. The accuracy of a membership condition is a probability that a cluster to which a provider belongs, determined on the basis of the second physiological information, the second questionnaire information, and the membership condition may match a cluster to which the provider belongs, determined by the clustering unit 102. As the accuracy increases, a matching probability becomes higher.

In FIG. 4, a transverse axis expresses the number of clusters generated by the clustering unit 102. In FIG. 4, a longitudinal axis expresses the accuracy of a membership condition. In FIG. 4, the maximum value of the accuracy of a membership condition is 1. In other words, in FIG. 4, in a case where the accuracy of a membership condition is 1, a cluster to which a provider belongs, determined on the basis of the second questionnaire information and the membership condition necessarily matches a cluster to which the provider belongs, determined by the clustering unit 102.

FIG. 4 shows that, in a case where the number of clusters generated by the clustering unit 102 is five or less, the accuracy of a membership condition is 0.7 or more and thus the accuracy is high.

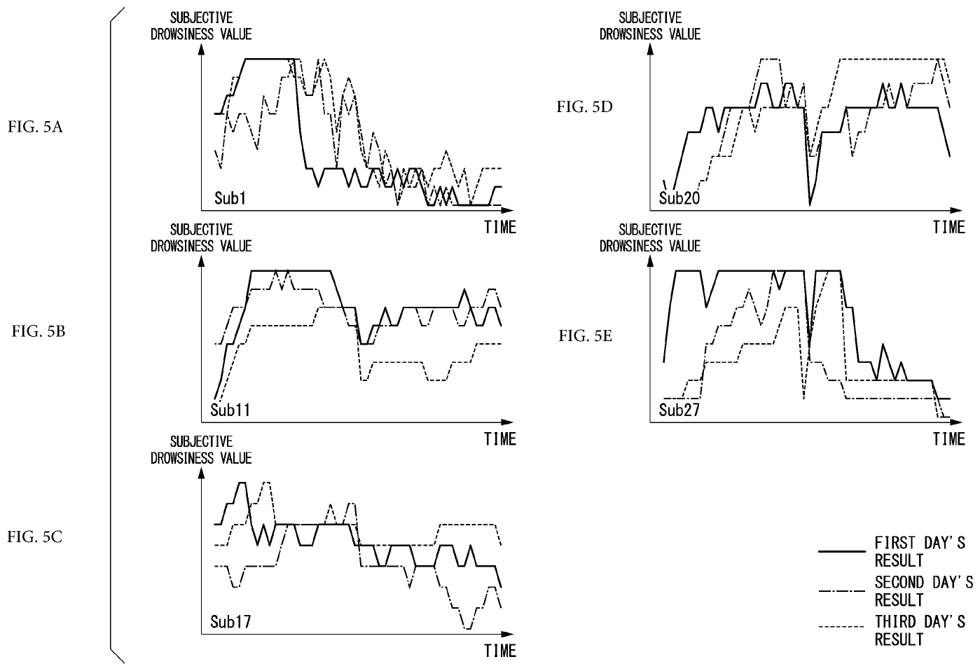

FIGS. 5A to 7D are diagrams of test results showing examples of the first questionnaire information in the embodiment. More specifically, FIGS. 5A to 7D show examples of test results in which a psychokinetic arousal level task test program (PVT test) is executed on 14 males 48 times per day over three days.

In other words, FIGS. 5A to 7D show examples of results of tests performed over separate three days. The PVT test was a test in which two sessions were executed per day and an arousal change due to caffeine intake was introduced in the middle. Right after each session, a subjective evaluation was performed on subject's own drowsiness/arousal in a questionnaire.

In the PVT test, the first physiological information acquired from the subject was electrocardiogram indexes HR, CSI, CVI, and HF/(LF+HF), a pulse wave, a pupil diameter, and motion of the head. HR indicates a heart rate RRᵢ for i minutes (where is an integer of 1 or greater). CSI indicates L/T in a plot diagram (hereinafter, Lorentz plot) regarding RRᵢ₊₁/RRᵢ. L indicates a variance on a diagonal axis in the Lorentz plot. T indicates a variance on an axis orthogonal to L.

CVI indicates Log₁₀(L×T). CVI indicates an integral value of power values in the section of 0.04 Hz<f<15 Hz in a power spectrum obtained by subjecting an electrocardiogram waveform to frequency space transform. The high frequency (HF) indicates an integral value of power values in the section of 0.224 Hz<f<0.28 Hz in a power spectrum in frequency analysis for a heartbeat change for seconds. The low frequency (LF) indicates an integral value of power values in the section of 0.04 Hz<f<0.12 Hz in a power spectrum in frequency analysis for a heartbeat change for seconds. HF/(LF+HF) indicates a ratio between LF (0.15 Hz<) and HF (0.15 Hz>).

A value indicating a pulse wave in a single PVT test is, for example, an average of amplitude values between pulse wave peaks in the single PVT test. A value indicating a pupil diameter in a single PVT test is, for example, an average value of pupil diameters in the single PVT test. A value indicating motion of the head in a single PVT test is, for example, an average value of movement amounts of the head in the single PVT test.

FIGS. 5A to 5E are first diagrams of test results showing examples of the first questionnaire information in the embodiment.

FIGS. 5A to 5E respectively show experimenters' own subjective evaluation results right after the PVT test is performed for persons allocated with identifiers such as “Sub1”, “Sub11”, “Sub17”, “Sub20”, and “Sub27” among the 14 male subjects.

In FIGS. 5A to 5E, a transverse axis expresses a test elapsed time. In FIGS. 5A to 5E, a longitudinal axis expresses a subjective drowsiness value. The subjective drowsiness value is one of values indicated by the first questionnaire information, and indicates that a subject feels drowsier as the value becomes greater.

Each of FIGS. 5A to 5E shows a test result for a first day, a test result for a second day, and a test result for a third day.

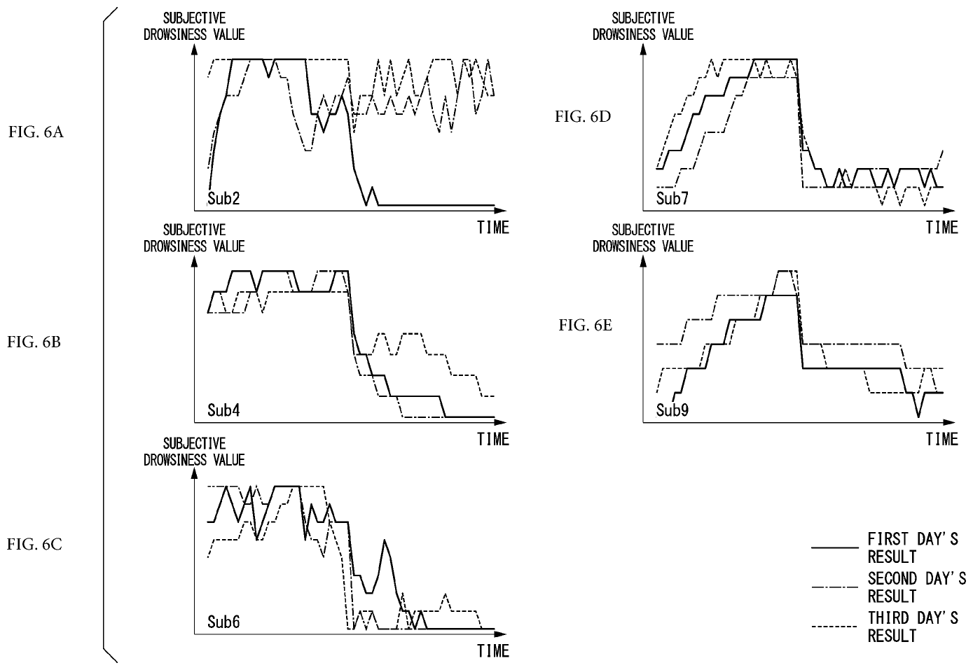

FIGS. 6A to 6E are second diagrams of test results showing examples of the first questionnaire information in the embodiment.

FIGS. 6A to 6E respectively show experimenters' own subjective evaluation results right after the PVT test is performed for persons allocated with identifiers such as “Sub2”, “Sub4”, “Sub6”, “Sub7”, and “Sub9” among the 14 male subjects.

In FIGS. 6A to 6E, a transverse axis expresses a test elapsed time. In FIGS. 6A to 6E, a longitudinal axis expresses a subjective drowsiness value.

Each of FIGS. 6A to 6E shows a test result for the first day, a test result for the second day, and a test result for the third day.

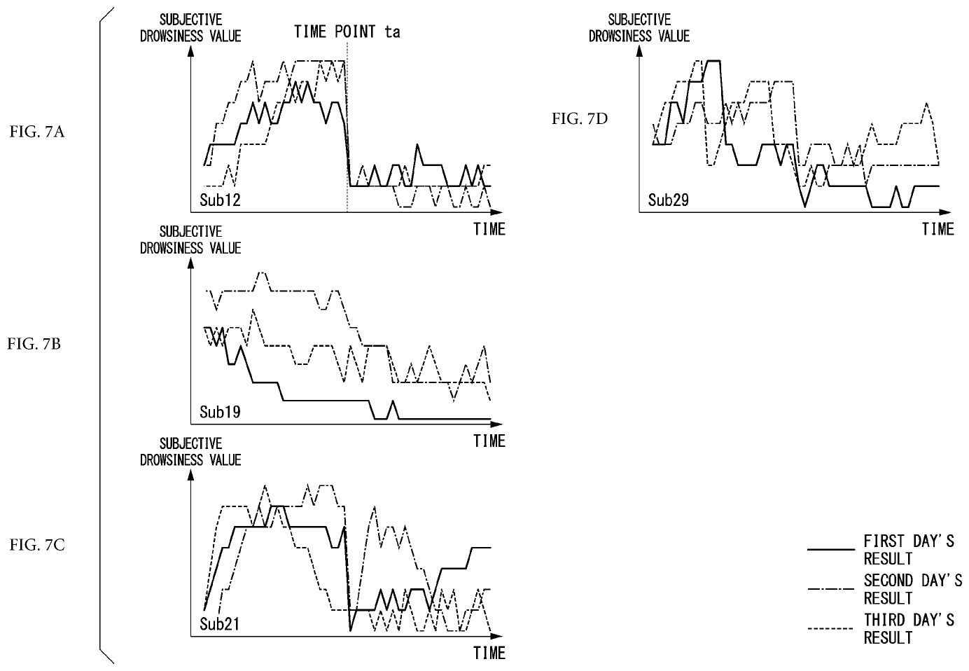

FIGS. 7A to 7D are third diagrams of test results showing examples of the first questionnaire information in the embodiment.

FIGS. 7A to 7D respectively show experimenters' own subjective evaluation results right after the PVT test is performed for persons allocated with identifiers such as “Sub12”, “Sub19”, “Sub21”, and “Sub29” among the 14 male subjects.

In FIGS. 7A to 7D, a transverse axis expresses a test elapsed time. In FIGS. 7A to 7D, a longitudinal axis expresses a subjective drowsiness value.

Each of FIGS. 7A to 7D shows a test result for the first day, a test result for the second day, and a test result for the third day.

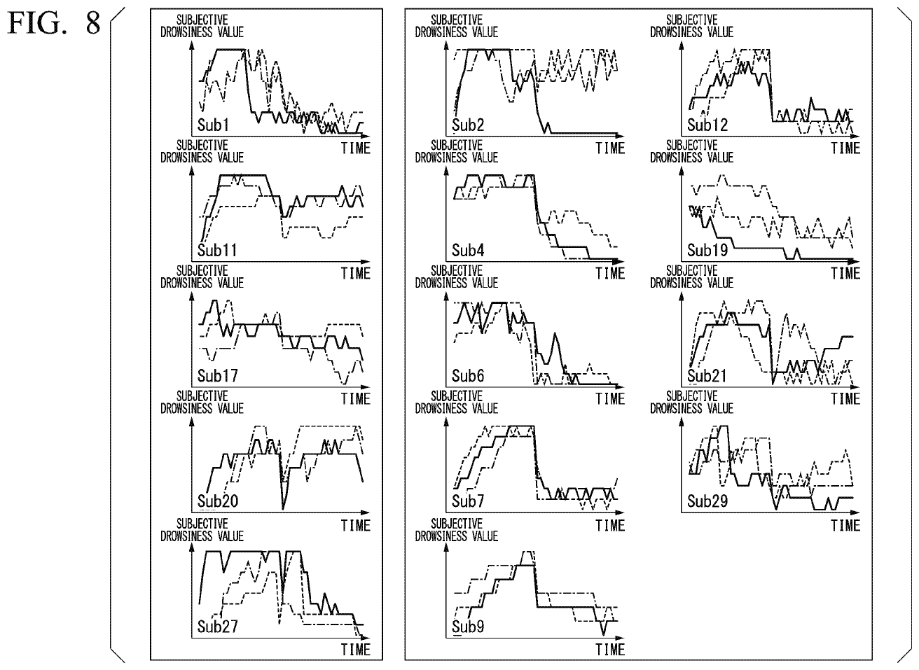

FIG. 8 is a diagram of test results showing examples of clustering process execution results in the embodiment.

FIG. 8 shows results of the state determination apparatus 1 performing clustering at the number of clusters k=1 to 8 by using the Bayzian-FDA method on the basis of the test results shown in FIGS. 5A to 7D. FIG. 8 shows that a Bayzian likelihood BIC is the minimum value at k=2 as a result of the state determination apparatus 1 performing clustering at k=1 to 8 by using the Bayzian-FDA method on the basis of the test results shown in FIGS. 5A to 7D. Here, k in the Bayzian-FDA indicates the number of clusters.

As shown in FIG. 8, the minimum value at k=2 indicates that the subjects corresponding to the test results in FIGS. 5A to 7D are classified into two clusters. FIG. 8 shows the fact that the subjects (that is, “Sub1”, “Sub11”, “Sub17”, “Sub20”, and “Sub27”) corresponding to the test results shown in FIG. 5 belong to one of the two clusters.

FIG. 8 shows the fact that the subjects (that is, “Sub2”, “Sub4”, “Sub6”, “Sub7”, “Sub9”, “Sub12”, “Sub19”, “Sub21”, and “Sub29”) corresponding to the test results shown in FIGS. 6A to 7D belong to the other cluster.

The accuracy of a state value estimated by a predetermined apparatus through machine learning on the basis of the test results shown in FIGS. 5A to 7D is as follows. In a case where a correlation coefficient was calculated with an error term as a binomial distribution by using a general linearized model, a correlation coefficient average in the state prediction/estimation for each subject was 0.44. On the other hand, in a case where a correlation coefficient in the state prediction/estimation for subjects belonging to a cluster was calculated in the same manner as in a separate cluster, R² was 0.53. As mentioned above, a correlation coefficient in the state prediction/estimation for subjects belonging to a cluster was calculated in the same manner as in a separate cluster, and thus the accuracy of estimating a state value was improved.

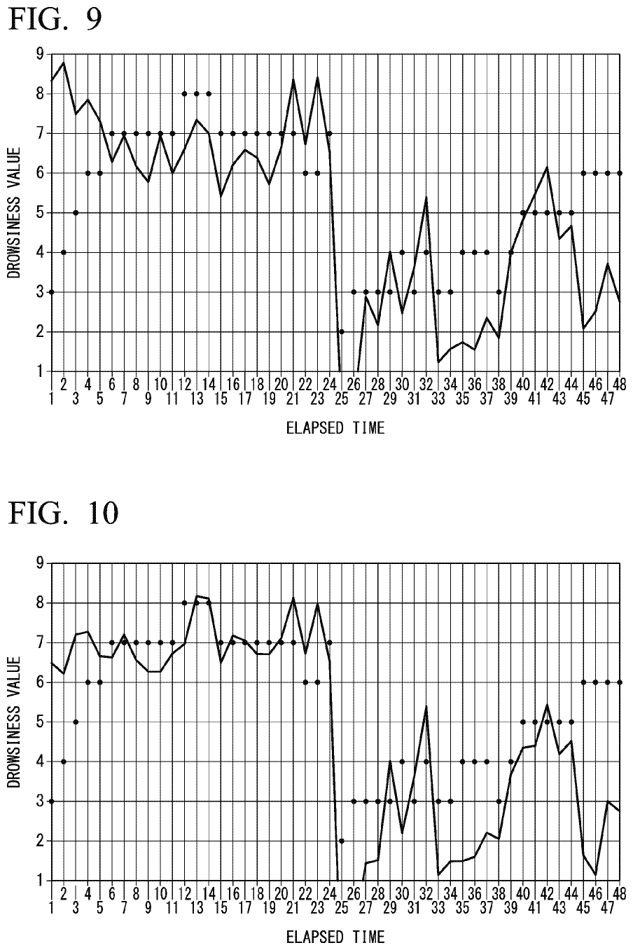

FIG. 9 is a first diagram of a test result showing an example of an execution result of the state information acquisition process in the embodiment.

FIG. 9 shows a result of the state determination apparatus 1 executing the state information acquisition process on the subject “Sub6” on the basis of the test result shown in FIG. 6C. In FIG. 9, a transverse axis expresses an elapsed time of the PVT test for the third day. In FIG. 9, a longitudinal axis expresses a drowsiness value. The drowsiness value is one of state values, and indicates that the drowsiness becomes stronger as the value becomes greater. The drowsiness value is a subjective evaluation value of Sub6, and a solid line indicates estimation results using a relational expression learned from all subjects other than Sub6. FIG. 9 shows the fact that the correlation coefficient R² in the state prediction/estimation is 0.41.

FIG. 10 is a second diagram of a test result showing an example of an execution result of the state information acquisition process in the embodiment.

FIG. 10 shows a result of the state determination apparatus 1 further adding a half of the test result shown in FIG. 6C to the test result shown in FIG. 6C and executing the state information acquisition process on the subject “Sub6”. In FIG. 10, a transverse axis expresses an elapsed time of the PVT test for the third day. In FIG. 10, a longitudinal axis expresses a drowsiness value. The drowsiness value is a subjective evaluation value of Sub6, and a solid line indicates estimation results using a relational expression learned from subjects excluding Sub6 from subjects in a cluster to which Sub6 belongs. FIG. 10 shows the fact that the correlation coefficient R² in the state prediction/estimation is 0.57.

Hitherto, FIGS. 9 and 10 have been described.

In the membership cluster determination process of determining to which one of the clusters shown in FIG. 8 the subject “Sub6” belongs, the state determination apparatus 1 executed a protocol such that subjective time-series features for the second day shown in FIGS. 5A to 7D can be partially extracted.

The second physiological information in the membership cluster determination process of determining to which one of the clusters shown in FIG. 8 the subject “Sub6” belongs was information indicating physiological information of a subject executing a simple task similar to the PVT test over a plurality of days.

The second physiological information in a simple task equivalent to the PVT test is information regarding physiological states of a subject executing the simple task equivalent to the PVT test over a plurality of days. The simple task equivalent to the PVT test was executed several times for a short time in time periods of different drowsiness in daily life such as the morning, the daytime, and the evening. The simple task equivalent to the PVT test was executed at rest.

The second physiological information acquired from the subject was electrocardiogram indexes HR, CSI, CVI, and HF/(LF+HF), a pulse wave, a pupil diameter, and motion of the head.

The protocol in the membership cluster determination process of determining to which one of the clusters shown in FIG. 8 the subject “Sub6” belongs was a protocol for determining that the subject belongs to a cluster having a stronger correlation.

Hitherto, the test results in FIGS. 5 to 10 have been described.

Here, detailed description will be made of a test when the test result in FIG. 2 is acquired. As the second physiological information, values calculated on the basis of physiology described below from physiological activities at rest for two minutes were used. In other words, the information was acquired according to a heartbeat analysis method by using values such as myoelectric potential activity states (an average activity amount and an activity fluctuation such as a deviation) of the corrugator supercilii muscle, the zygomatic muscle, and the laughing muscle, an average respiratory frequency and a fluctuation such as a frequency deviation, a blink frequency, an average, a deviation, the maximum value, and the minimum value of a skin resistance level, averages, deviations, the maximum values, and the minimum values of a baseline and amplitude in data output from fingertip PPG sensors, an RRI average, a standard deviation, the maximum, the minimum in heartbeat information, and SSD, NN, Pnn, LF, HF, CVI, and CSI. As the simple questionnaire information, BIS/BAS (behavior control), STAI-trait (anxiety characteristic), STAI-state (anxiety state), IRI (sympathy), BDI (depression), TAS (apathy), ERQ/EQ (emotional control), PSQI (sleep habit), and Emphasizing Quotient/Systemizing Quotient, Japanese version were used. Clusters were estimated by using the decision tree on the basis of the pieces of simple information, and a result of four pieces of simple data contributing to estimation of the clusters was obtained in estimation of five clusters.

Hitherto, details of the test when the test result in FIG. 2 is acquired have been described.

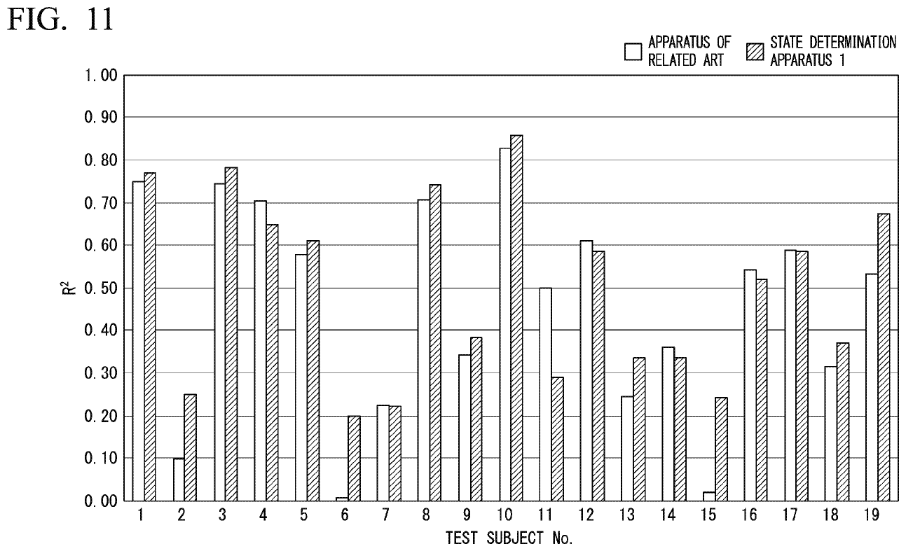

FIG. 11 is a diagram showing an example of a test result showing the accuracy of the state determination apparatus 1 of the embodiment determining a state of a determination target person and the accuracy of an apparatus of the related art determining a state of a determination target person.

In FIG. 11, a transverse axis expresses a subject. In FIG. 11, a longitudinal axis expresses a value of the correlation coefficient R² in the state prediction/estimation. FIG. 11 shows a value of the correlation coefficient R² in the state prediction/estimation in the apparatus of the related art and a value of the correlation coefficient R² in the state prediction/estimation in the state determination apparatus 1 for each subject. FIG. 11 shows that an average value of the correlation coefficient R² in the state prediction/estimation is 0.45 in the apparatus of the related art. FIG. 11 shows that an average value of the correlation coefficient R² in the state prediction/estimation is 0.51 in the state determination apparatus 1. FIG. 11 shows that, although there is an individual difference, a value of the correlation coefficient R² in the state prediction/estimation is greater in the state determination apparatus 1 than in the apparatus of the related art with respect to a large number of subjects.

The state determination apparatus 1 of the embodiment configured as described above determines to which cluster a determination target person belongs among a plurality of clusters that are results clustered in advance on the basis of the first physiological information and the first questionnaire information provided by population constituents. The state determination apparatus 1 acquires state information that is information indicating a state of the determination target person on the basis of state relationship information of each cluster indicated by a determination result. The state determination apparatus 1 determines a state of the determination target person on the basis of the acquired state information. As mentioned above, the state determination apparatus 1 does not determine a state of a person according to a one-dimensional method, but determines a state of a determination target person according to a state equation corresponding to a cluster to which the determination target person belongs. Thus, the state determination apparatus 1 can suppress deterioration in determination accuracy due to an individual difference in a change of a state of a person.

The state determination apparatus 1 of the embodiment configured as described above executes the clustering process, the state relationship information acquisition process, the membership condition acquisition process, the membership cluster determination process, and the state information acquisition process. Thus, the state determination apparatus 1 of the embodiment configured as described above requires the time for the clustering process. Since the clustering method requires less time than the machine learning, the clustering process requires less time than the machine learning. Thus, the state determination apparatus 1 of the embodiment configured as described above can determine a state of a determination target person for a time equal to or shorter than the time required to determine a state of the determination target person according to the machine learning method.

The state determination apparatus 1 of the embodiment as described above executes the clustering process on the basis of updated physiological information and updated questionnaire information even after a state value is calculated.

Hereinafter, the clustering process after state information is acquired will be referred to as a re-clustering process. The re-clustering process is the process in step S210. Thus, the state determination apparatus 1 of the embodiment configured as described above can determine a state of a person with higher accuracy after the re-clustering process is executed than before the re-clustering process is executed.

The state determination apparatus 1 of the embodiment configured as described above updates a value of a coefficient of state relationship information on the basis of state information, and the first physiological information of a determination target person. Hereinafter, a process of updating the state information after the state information is acquired will be referred to as a state information update process. The state information update process is the process in step S209. Thus, the state determination apparatus 1 of the embodiment configured as described above can determine a state of a person with higher accuracy after the state information update process is executed than before the state information update process is executed.

### Modification Example

The first physiological information, the second physiological information, the first questionnaire information, and the second questionnaire information can be preferably information acquired in a situation in which a state to be determined by the state determination apparatus 1 is changed with a meaningful difference.

For example, in a case where a state to be determined is drowsiness, the first physiological information, the second physiological information, the first questionnaire information, and the second questionnaire information can be preferably acquired in time periods of different drowsiness in daily life such as the morning, the daytime, and the evening.

In a case where a large load is applied to a subject, a physiological state of the subject may be changed in a complex manner by the large load. Thus, the first physiological information and the second physiological information cannot be preferably information acquired after a large load is applied to a subject. For example, in a case where a state to be determined is comfort/discomfort of a person, the first physiological information and the second physiological information, and the first questionnaire information and the second questionnaire information can be preferably acquired when a provider feels a stimulus that a person feels comfortable or uncomfortable. The stimulus that a person feels comfortable or uncomfortable is, for example, an image, music, touch, smell, or taste. The first physiological information and the second physiological information, and the first questionnaire information and the second questionnaire information can be preferably information acquired with respect to various stimuli from comfort to discomfort. Loads of the stimuli may be a small load to a large load.

All or some of the functions of the state determination apparatus 1 may be realized by using hardware such as an application specific integrated circuit (ASIC), a programmable logic device (PLD), or a field programmable gate array (FPGA). A program may be recorded on a computer readable recording medium. The computer readable recording medium is, for example, a portable medium such as a flexible disk, a magnetooptical disc, a ROM, or a CD-ROM, and a storage device such as a hard disk built into a computer system. The program may be transmitted via an electric communication line.

An information amount of the second physiological information may be equal to or smaller than that of the first physiological information. An information amount of the second questionnaire information may be equal to or smaller than that of the first questionnaire information.

A membership condition may not necessarily be a condition based on both of the second physiological information and the second questionnaire information. In this case, the acquisition unit 101 may acquire only information on which a membership condition is based of the second physiological information and the second questionnaire information. In other words, the acquisition unit 101 may acquire only the second physiological information, and may acquire only the second questionnaire information.

The first update unit 109 is an example of a state relationship update unit. The state information acquisition unit 106 is an example of an answer estimation unit.

The state determination apparatus 1 may be installed by a plurality of information processing apparatuses communicably connected to each other via a network. In this case, the respective functional units of the state determination apparatus 1 may be installed to be distributed to the plurality of information processing apparatuses. For example, the clustering unit 102 and the state relationship information acquisition unit 103, and the other functional units may be installed in different information processing apparatuses.

While preferred embodiments of the invention have been described and shown above, it should be understood that these are exemplary of the invention and are not to be considered as limiting. Additions, omissions, substitutions, and other modifications can be made without departing from the scope of the present invention. Accordingly, the invention is not to be considered as being limited by the foregoing description, and is only limited by the scope of the appended claims.

What is claimed is:

1. A state determination apparatus comprising:
an acquisition unit acquiring first physiological information, and second physiological information or second questionnaire information with respect to a determination target person who is a determination target among first questionnaire information that is information indicating an answer of an answerer to a first questionnaire in which the answer does not indicate an individuality of the answerer, the first physiological information that is information indicating a physiological state of the answerer to the first questionnaire and not indicating the individuality of the answerer to the first questionnaire, the second questionnaire information that is information indicating an answer of an answerer to a second questionnaire in which the answer indicates an individuality of the answerer, and the second physiological information that is information indicating a physiological state of the answerer to the second questionnaire and indicating an individuality of the answerer to the second questionnaire;
a membership cluster estimation unit estimating to which cluster the determination target person belongs among a plurality of clusters forming a population of answerers based on the second physiological information or the second questionnaire information, and a membership condition according to the second physiological information or the second questionnaire information;
an answer estimation unit estimating an answer of the determination target person to the first questionnaire based on state relationship information indicating a relationship between the first questionnaire information and the first physiological information, predefined for each of the plurality of clusters, the state relationship information corresponding to an estimation result of the membership cluster estimation unit, and the first physiological information provided by the determination target person; and
a state determination unit determining a state of the determination target person based on the answer estimated by the answer estimation unit,
wherein the first physiological information and the first questionnaire information are information indicating an individuality through a combination of the first physiological information and the first questionnaire information.

2. The state determination apparatus according to claim 1, further comprising:
a state relationship information acquisition unit acquiring state relationship information for each cluster based on the first physiological information and the first questionnaire information provided by a plurality of population constituents each forming the population; and
a first update unit updating the state relationship information based on the answer and the first physiological information provided by the determination target person in a case where an update condition that is a predetermined condition related to the answer estimated by the answer estimation unit is satisfied.

3. The state determination apparatus according to claim 2, further comprising:
a clustering unit classifying the population constituents into a plurality of clusters based on the first physiological information and the first questionnaire information of the plurality of population constituents; and
a second update unit causing the clustering unit to classify the population constituents and the determination target person into a plurality of clusters based on the first physiological information and the first questionnaire information of the plurality of population constituents, the first physiological information of the determination target person, and the state relationship information corresponding to an estimation result of the membership cluster estimation unit, in a case where the update condition is satisfied.

4. A state determination method comprising:
acquiring first physiological information, and second physiological information or second questionnaire information with respect to a determination target person who is a determination target among first questionnaire information that is information indicating an answer of an answerer to a first questionnaire in which the answer does not indicate an individuality of the answerer, the first physiological information that is information indicating a physiological state of the answerer to the first questionnaire and not indicating the individuality of the answerer to the first questionnaire, the second questionnaire information that is information indicating an answer of an answerer to a second questionnaire in which the answer indicates an individuality of the answerer, and the second physiological information that is information indicating a physiological state of the answerer to the second questionnaire and indicating an individuality of the answerer to the second questionnaire;
estimating to which cluster the determination target person belongs among a plurality of clusters forming a population of answerers based on the second physiological information or the second questionnaire information, and a membership condition according to the second physiological information or the second questionnaire information;
estimating an answer of the determination target person to the first questionnaire based on state relationship information indicating a relationship between the first questionnaire information and the first physiological information, predefined for each of the plurality of clusters, the state relationship information corresponding to a result of the estimation regarding a membership cluster, and the first physiological information provided by the determination target person; and
determining a state of the determination target person based on the estimated answer,
wherein the first physiological information and the first questionnaire information are information indicating an individuality through a combination of the first physiological information and the first questionnaire information.

5. A non-transitory computer readable recording medium including a computer program for causing a computer to function as the state determination apparatus according to claim 1.
